(19) United States
(12) Patent Application Publication
(10) Pub. No.: US 2007/0288084 A1
(43) Pub. Date: Dec. 13, 2007

(54) Implantable Stent with Degradable Portions

(75) Inventors: Michael J. Lee, Santa Rosa, CA (US); Richard L. Klein, Santa Rosa, CA (US)

Correspondence Address: KIRKPATRICK & LOCKHART PRESTON GATES ELLIS LLP;ATTN: JOSEPH TAFFY, 1900 MAIN STREET, SUITE 600, IRVINE, CA, 92614-7319, US

(73) Assignee: MEDLOGICS DEVICE CORPORATION, Santa Rosa, CA (US)

(21) Appl. No.: 11/423,396

(22) Filed: Jun. 9, 2006

(51) Int. Cl. A61F 2/06

(52) U.S. Cl. 623/1.16; 623/1.34; 623/1.38; 623/1.42

(57) ABSTRACT

Disclosed herein are stents with degradable part and/or sections of the stent. The degradable portions of the disclosed stents can provide increased surface area for a period of time for bioactive material deposition or enhanced radiopacity at various locations along the length of the stent. The stents of the present invention also can stent portions of a vessel that are damaged by stent implantation but do not necessarily require long term stenting by providing degradable end sections on the stents of the present invention.

### FIELD OF THE INVENTION

This invention relates to an improved implantable stent for the treatment, inhibition and/or prevention of restenosis, abrupt reclosure or reocclusion in coronary or peripheral vessels. More specifically, the invention relates to an improved implantable stent with degradable portions. The degradable portions of the stents of the present invention provide for, among other things, the delivery and release of bioactive materials, enhanced radiopacity and/or the treatment of portions of vessels damaged by stent implantation that do not necessarily require long-term stenting.

### BACKGROUND OF THE INVENTION

Cardiovascular disease, including atherosclerosis, is the leading cause of death in the United States. The medical community has developed a number of methods and devices for treating coronary disease, some of which are specifically designed to treat the complications resulting from atherosclerosis and other forms of coronary artery narrowing.

An important development for treating atherosclerosis and other forms of vascular narrowing is percutaneous transluminal angioplasty, and, in the specific instance of coronary artery disease, percutaneous transluminal coronary angioplasty, hereinafter collectively referred to as “angioplasty.” The objective of angioplasty is to enlarge the lumen (inner tubular space) of an affected vessel by radial hydraulic expansion. The procedure is accomplished by inflating a balloon within the narrowed lumen of the affected vessel. Radial expansion of the affected vessel occurs in several different dimensions, and is related to the nature of the plaque narrowing the lumen. Soft, fatty plaque deposits are flattened by the balloon, while hardened deposits are cracked and split to enlarge the lumen. The wall of the vessel itself is also stretched when the balloon is inflated.

Unfortunately, while the affected vessel can be enlarged thus improving blood flow, in some instances the vessel recloses chronically (“restenosis”), closes down acutely (“abrupt reclosure”) or reoccludes (all hereinafter referred to as “reclosure”), negating the positive effect of the angioplasty procedure. Such reclosure frequently necessitates repeat angioplasty or open heart surgery. While such reclosure does not occur in the majority of cases, it occurs frequently enough that such complications comprise a significant percentage of the overall failures of the angioplasty procedure, for example, twenty-five to thirty-five percent of such failures.

To lessen the risk of reclosure, various devices have been proposed for mechanically keeping the affected vessel open after completion of the angioplasty procedure. Such endoprostheses (generally referred to as “stents”), are typically inserted into the vessel, positioned across the lesion or stenosis, and then expanded to keep the passageway clear. The stent provides a scaffold which overcomes the natural tendency of the vessel walls of some patients to reclose, thus maintaining the patency of the vessel and resulting blood flow.

Some stents release bioactive materials, such as drugs, to reduce the risk of reclosure. These stents have been somewhat successful at aiding in the treatment, inhibition and/or prevention of reclosure. However, the small size and intricate design and configuration of stents have limited the amount of bioactive materials (and additionally the choice of bioactive materials) that can be successfully loaded onto the device. Thus, a need exists for stent designs and configurations that increase a stent's ability to carry and release bioactive materials at a treatment site.

Further, in some instances, balloon expansion expands portions of a vessel beyond the ends of the implantable stent. These portions of the vessel, therefore, are damaged by the implantation procedure but do not receive the therapeutic benefits of the implanted stent. Thus, a need also exists for methods and devices that treat these portions of a vessel that have been damaged by stent implantation but do not necessarily require long-term stenting.

Finally, due to their generally straight and tubular shape, conventional stents are most effective at restoring patency of vessels when the area to be treated is a uniform and relatively straight area of the vessel. Vessels, however, branch numerous times as they travel throughout the body. When a vessel branches, the opening to the branched vessel is called an ostium. Often, when treatment is required both before and after an ostium, this opening is “gated” by the stent passing over it. Such gating generally is not acceptable because it impedes blood flow into the vessel branch. If the ostium is not gated in this manner, however, treatment before and after the vessel branch is often incomplete. Based on these described issues, there is room for improvement in the design and use of stents. The presently disclosed invention provides such improvements.

### SUMMARY OF THE INVENTION

The present invention provides solutions to problems identified with currently-available stents. First, the stents of the present invention incorporate novel designs and configurations that can increase the available surface area of the stent for radiopacity during positioning of the stents and/or bioactive material deposition. Increased surface area for bioactive material deposition aids in the treatment, inhibition and/or prevention of restenosis, abrupt reclosure and/or reocclusion (all hereinafter referred to as “reclosure”). These portions of the stent that increase surface area for radiopacity and/or bioactive material deposition are degradable so that they do not add long-term bulk to the stent once the positioning of the stent is complete or the release of bioactive materials is no longer required. Embodiments of the present invention can adopt one or more degradable sections at one or more ends of the stent that treat portions of a vessel that would otherwise be damaged and left untreated by stent implantation. In another embodiment of the present invention, one or more degradable tabs can be included at any location within the stent. The degradable sections treat these portions of the vessel for a time and eventually erode to leave the portions unstented. These degradable sections can also contain and release bioactive materials to aid in the prevention, treatment and/or inhibition of reclosure. Degradable portions can also be found at other locations of the stent. For instance, when a stent is placed at a vessel branch, the portion of the stent that might otherwise gate the ostium of the branch can be quickly degradable to avoid this problem.

Specifically, one embodiment according to the present invention is a stent comprising a first end portion, a central portion and a second end portion; wherein the first end portion, the central portion and the second end portion each comprise one or more sections; wherein each of the one or more sections comprises a series of struts and crowns; and wherein at least a part of the one or more sections is degradable.

Locations of degradable parts, portions or sections can be selected from the group consisting of within the first end portion; within the central portion; within the second end portion; and combinations thereof. In one embodiment, one or more sections in the first end portion are degradable. In another embodiment, one or more sections adjacent to the one or more degradable sections in the first end portion are also degradable. When the first end portion comprises degradable sections, one or more of the one or more sections of the second end portion can also be degradable. Further, when the second end portion comprises degradable sections, one or more sections adjacent to the one or more degradable sections of the second end portion can also be degradable. In one embodiment, the central portion is non-degradable.

Degradable parts, sections or portions of the present invention can comprise a bioactive material, a radiopacity enhancing material, or both. Degradable parts can also be made of or can comprise tabs that can include a bioactive material, a radiopacity enhancing material, or both. Tabs can be found within end crowns anywhere along the length of the stent and can also extend from end crowns longtitudinally away from the stent at one or both end portions.

The present invention also includes endoprosthetic devices. One endoprosthetic device according to the present invention comprises a plurality of segments including a proximal end segment, a distal end segment, at least one intermediate segment between the proximal end segment and the distal end segment and a degradable disc. The degradable disc can include a bioactive material, a radiopacity enhancing material, or both. Endoprosthetic devices according to the present invention can also comprise a plurality of degradable discs, some or all of which can include a bioactive material, a radiopacity enhancing material, or both.

In another embodiment of an endoprosthetic device according to the present invention the plurality of segments are arranged along and define a longitudinal axis; and the proximal end segment has a series of crowns, struts and end crowns and further wherein at least one end crown has a rim extending longitudinally away from the at least one intermediate segment and wherein the rim is adapted to receive a degradable disc. In another embodiment, the distal end segment also has a series of crowns, struts and end crowns and further wherein at least one distal segment end crown has a rim extending longitudinally away from the at least one intermediate segment and wherein the rim is adapted to receive one of a plurality of degradable discs.

### BRIEF DESCRIPTION OF THE DRAWINGS

FIG. 1 depicts an embodiment of the present invention including degradable sections with two embodiments of the tabs of the present invention.

FIG. 2 shows an alternative embodiment of the present invention including degradable sections with two embodiments of the tabs of the present invention.

FIG. 3 depicts an embodiment of the present invention including degradable tabs located in different sections of a stent. This depicted embodiment does not include degradable sections.

FIG. 4 depicts a rim of the present invention within an end crown and a rim of the present invention within an end crown with a degradable disc inserted within the rim.

FIG. 5 depicts degradable sections, one of which includes an embodiment of the degradable tabs of the present invention.

FIGS. 6A and 6B depict end portions of stents with additional degradable portion configurations.

FIG. 7 depicts various methods through which degradable sections according to the present invention can be attached to nondegradable portions.

### DEFINITION OF TERMS

The term “stents” refers to devices that are used to maintain patency of a body lumen or interstitial tract. There are two categories of stents; those which are balloon expandable (e.g., stainless steel) and those which are self-expanding (e.g., nitinol). Stents are currently used in peripheral, coronary, and cerebrovascular vessels, the alimentary, hepatobiliary, and urologic systems, the liver parenchyma (e.g., porto-systemic shunts), and the spine (e.g., fusion cages). In the future, stents will be used in smaller vessels (currently minimum stent diameters are limited to about 2 millimeters). For example, they will be used in the interstitium to create conduits between the ventricles of the heart and coronary arteries, or between coronary arteries and coronary veins. In the eye, stents are being developed for the Canal of Schlem to treat glaucoma.

As used herein the phrase, “bioactive materials” refers to any organic, inorganic, or living agent that is biologically active or relevant. For example, a bioactive material can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive materials also can include drugs such as chemical or biological compounds that can have a therapeutic effect on a biological organism. Bioactive materials include those that are especially useful for long-term therapy such as hormonal treatment. Examples include drugs for contraception and hormone replacement therapy, and for the treatment of diseases such as osteoporosis, cancer, epilepsy, Parkinson's disease and pain. Suitable biological materials can include, e.g., anti-inflammatory agents, anti-infective agents (e.g., antibiotics and antiviral agents), analgesics and analgesic combinations, antiasthmatic agents, anticonvulsants, antidepressants, antidiabetic agents, antineoplastics, anticancer agents, antipsychotics, and agents used for cardiovascular diseases such as anti-restenosis and anti-coagulant compounds. Exemplary drugs include, but are not limited to, antiproliferatives such as paclitaxel and rampamycin, everolimus, tacrolimus, des-aspartate angiotensin 1, exochelins, nitric oxide, apocynin, gamma-tocopheryl, pleiotrophin, estradiol, heparin, aspirin and 5-hydroxy-3-methylglutaryl-coenzyme A reductase (HMG-CoA) reductase inhibitors such as atorvastatin, cerivastatin, fluvastatin, lovastatin, pravastatin, rosuvastatin, simvastatin, etc.

Bioactive materials also can include precursor materials that exhibit the relevant biological activity after being metabolized, broken-down (e.g. cleaving molecular components), or otherwise processed and modified within the body. These can include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive materials within the intended meaning herein. Aspects of the present invention directed toward bioactive materials can include any or all of the foregoing examples.

As used herein, the term “degradable” includes a feature of a material that allows its complete or near complete disappearance from an area over time. In addition to the conventional meaning of degradable, this term should also be read to include erodable and absorbable materials.

### DETAILED DESCRIPTION OF THE INVENTION

U.S. Pat. Nos. 5,292,331 and 5,135,536 to Boneau and Hilstead respectively, and the references cited therein, make it clear that stents can be configured and constructed in many different ways. The present invention is applicable to all known stent configurations, and it will be readily apparent from the following discussion of several exemplary configurations how the invention can be applied to any other type of stent construction.

As stated earlier, it is often beneficial for an implanted stent to release a bioactive material to reduce the physiological trauma associated with the stent's implantation and to aid in the treatment, inhibition and/or prevention of restenosis, abrupt reclosure or re-occlusion (all hereinafter referred to as “reclosure”). The stents of the present invention are designed to allow for varying amounts of bioactive material release along the stent due to increased surface area of various portions of the stent. These portions of the stent that increase surface area for bioactive material deposition are degradable so that they do not add long term bulk to the stent once the release of bioactive materials is no longer required. The increased surface area can be provided anywhere along the length of the stent. In one embodiment, the increased surface area is provided at the ends of the stent to provide more bioactive material release at the ends. Providing for increased release of bioactive materials at the ends of a stent can be beneficial to ease the transition between stented and unstented portions of a vessel and to also provide therapeutic benefit to those portions of the vessel damaged during the implantation procedure, but beyond the ends of the stent.

Stents of the present invention also can help treat areas of a vessel that are damaged by stent implantation that do not necessarily require long-term stenting. The present invention treats these areas by including degradable sections at the ends of the stent. These degradable sections can contain bioactive materials and can release these bioactive materials at damaged portions of the vessel, stent them for a time, and eventually erode, leaving a non-stented portion of the vessel. The present invention also can include degradable portions with or without bioactive materials at other locations. For instance, when a stent is placed a vessel branch, the portion of the stent that might otherwise gate the ostium of the branch can be quickly degradable to avoid this problem. As stated, described degradable portions of the present invention can include bioactive materials and/or radiopaque materials to aid in device implantation. In one embodiment, the majority of the stent can be degradable with only a portion remaining for long term treatment site identification.

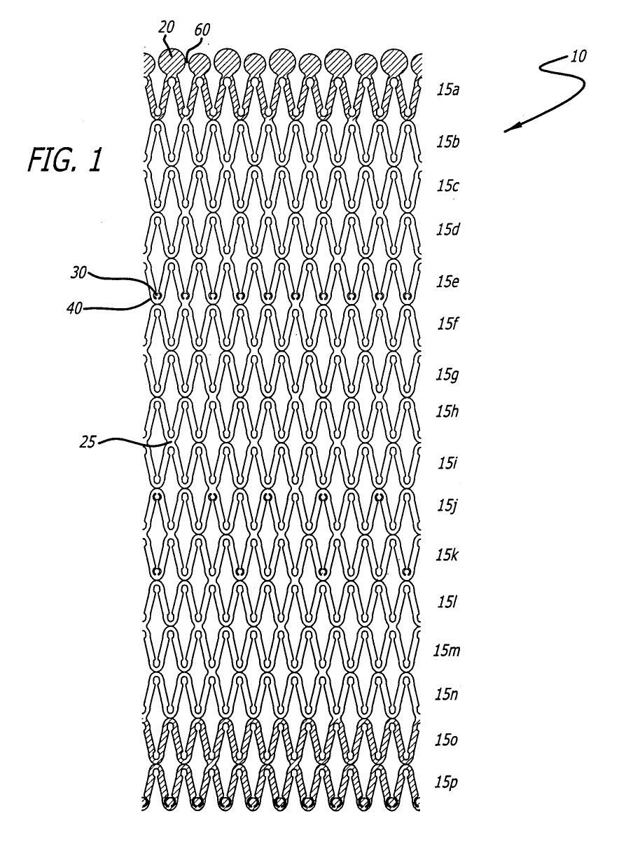

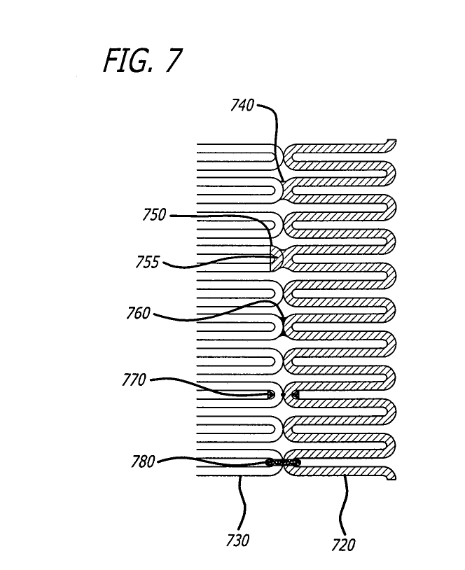

FIG. 1 depicts one embodiment of the stents of the present invention. The stents of the present invention may have more or less undulations than are shown in FIG. 1 (and generally similar subsequent FIGS.), but the simplified depictions shown herein will be sufficient to illustrate the present invention. In FIG. 1, the depicted stent 10 includes 16 “sections,” 15a-p While these “sections” of the stent are referred to as such for ease of description, one should note that the sections 15b-15n are not formed separately, but instead are continuous and joined by “crossovers” 25. The degradable sections, including sections 15a, 15o and 15p, are adhered to the main body of the stent through techniques known to those of ordinary skill in the art, several examples of which are described in more detail in relation to FIG. 7. Further, while this FIG. 1 depicts 16 repeating sections, it is to be understood that the stents of the present invention can include more or less than 16 sections.

The embodiment of the stents of the present invention depicted in FIG. 1 includes degradable sections 15a, 15o and 15p. Degradable segment 15a includes one embodiment of the degradable tabs 20 of the present invention. These tabs 20 are continuous with and extend from the ends of the terminal end crowns 60. Thus, these tabs 20 extend the length of the stent 10. Depending on whether these tabs 20 adopt the same width as the crowns 60 of a particular embodiment, these tabs 20 may or may not affect the crossing profile of the stent 10. If the tabs 20 maintain the same width or have a smaller width than the crowns 60, the crossing profile will not be affected even if every end crown 60 comprises such a tab 20. The tabs 20 of the present invention can have widths larger than the crowns 60 of a particular embodiment and, as a result, would affect the crossing profile if a tab 20 were to be included on every end crown 60. Including tabs 20 with widths larger than the width of crowns 60 of a particular embodiment may not affect the crossing profile, however, if such tabs 20 are included on less than every end crown 60 or are configured to overlap with one another.

In FIG. 1, sections 15e, 15j, 15k and 15p include a degradable tab embodiment of the present invention. These degradable tabs 30 are found within crowns 40 of the stents of the present invention. These degradable tabs 30 provide additional surface area for bioactive material deposition without affecting the length or crossing profile of the stent. These depicted degradable tabs 30 demonstrate that the degradable tabs of the present invention can be included anywhere along the length of the stent. Further, these degradable tabs 30 may be included in every crown of a particular section or any other combination of crowns in a given section, including every other crown, every third crown, every fourth crown, a random distribution, etc. The degradable tabs of the present invention increase surface area for enhanced radiopacity and/or increased area for bioactive material deposition. Bioactive materials can be found within the material comprising the degradable tabs or can be deposited onto the surface of the degradable tabs.

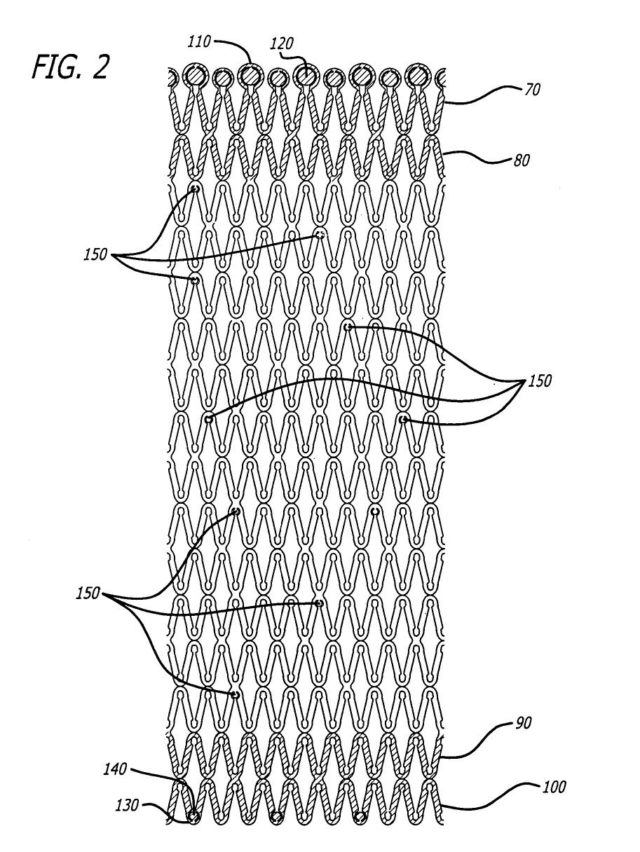

FIG. 2 depicts another embodiment of the stents of the present invention. In this embodiment, each end of the stent contains two degradable sections, such that the stent has four degradable sections 70, 80, 90 and 100. Specifically, the last sections 70 and 100 of the stent are degradable as well as the stent sections 80 and 90 that are adjacent to sections 70 and 100, respectively. In the embodiment of the stents of the present invention depicted in FIG. 2, every end crown 110 of degradable section 70 contains a tab 120, while every third crown 130 of degradable section 100 contains a tab 140. Degradable tabs 150 also are found in crowns located throughout the length of the stent.

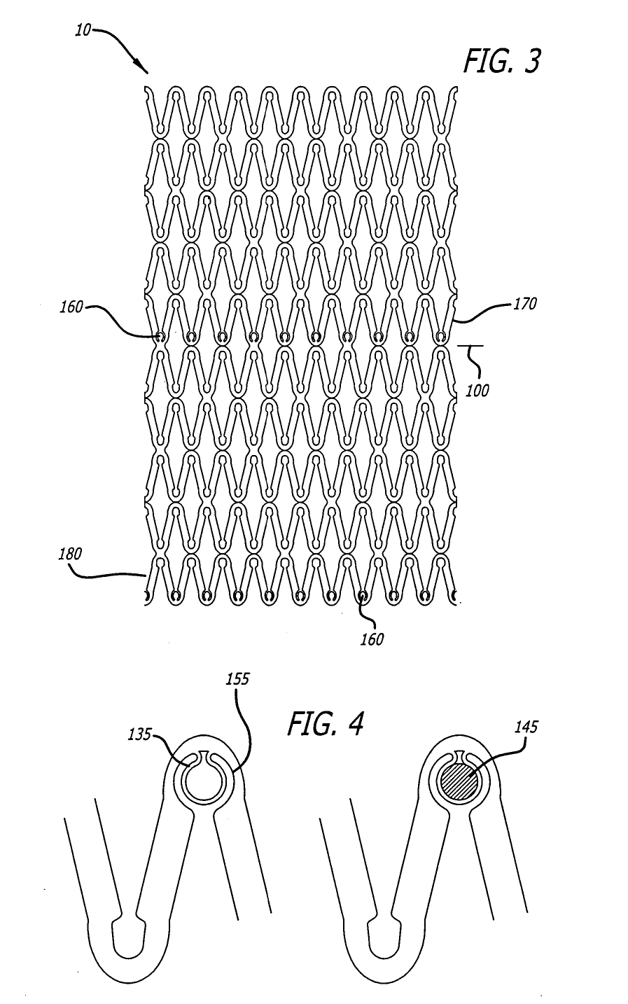

FIG. 3 depicts another embodiment of the stents of the present invention. This embodiment of the stents of the present invention does not include degradable sections. Instead, this embodiment of the stents of the present invention includes degradable tabs 160 found in crowns in two different sections 170 and 180 of the stent. This configuration of degradable tab placement can be useful when the tabs 160 are included to aid in radiopacity during positioning of the stent. Degradable tabs 160 may be useful towards the middle of a stent when, for example, the stent is to be positioned in the area of a vessel branch or bifurcation. While not depicted, radiopacity enhancing materials on a degradable portion within the stent can be positioned at a vessel branch opening (ostium) so that this portion of the stent can be precisely placed at the ostium and will degrade to avoid long term gating of the vessel branch. Degradable tabs 160 at the end of a stent 180 can be useful for positioning start and/or end positions of the stent.

FIG. 4 depicts an alternative embodiment of the stents of the present invention in which a nondegradable framework can be filled in with a degradable material. In this depicted embodiment, a rim 135 of the same material of the body of the stent is cut within a crown 155. In this embodiment, a degradable material 145 (in one embodiment a degradable material containing a bioactive material and/or a radiopacity enhancing material) can be appropriately-shaped to be fit into the rim 135. The rim 135 and appropriately-shaped degradable material 145 can be any shape that fits within a crown 155 of the stent. In one embodiment, the degradable material 145 can be press fit into the rim 135. Degradable materials can also be sprayed into the rim, dip-coated into the rim or placed into the rim according to any other appropriate method known to those of ordinary skill in the art.

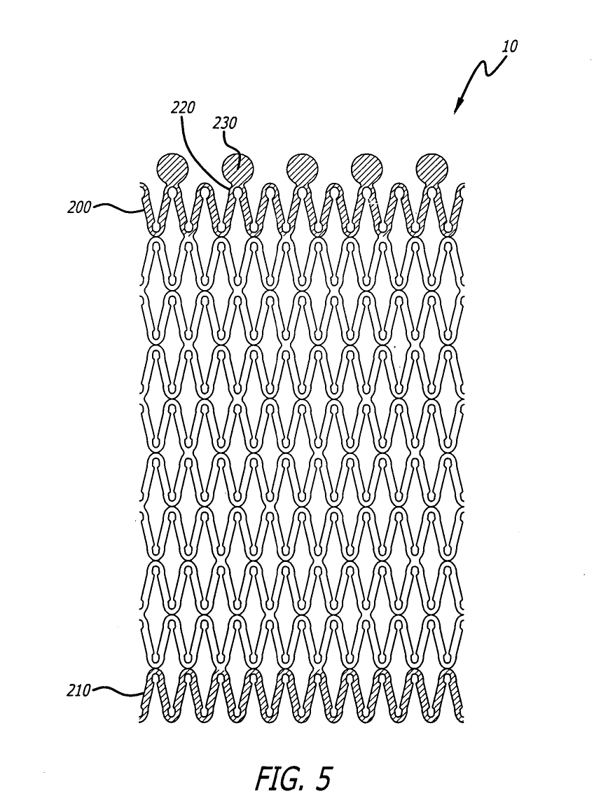

FIG. 5 depicts another embodiment of the stents of the present invention. This depicted embodiment includes degradable sections 200 and 210. Every other terminal crown 220 of degradable section 200 comprises a degradable tab 230.

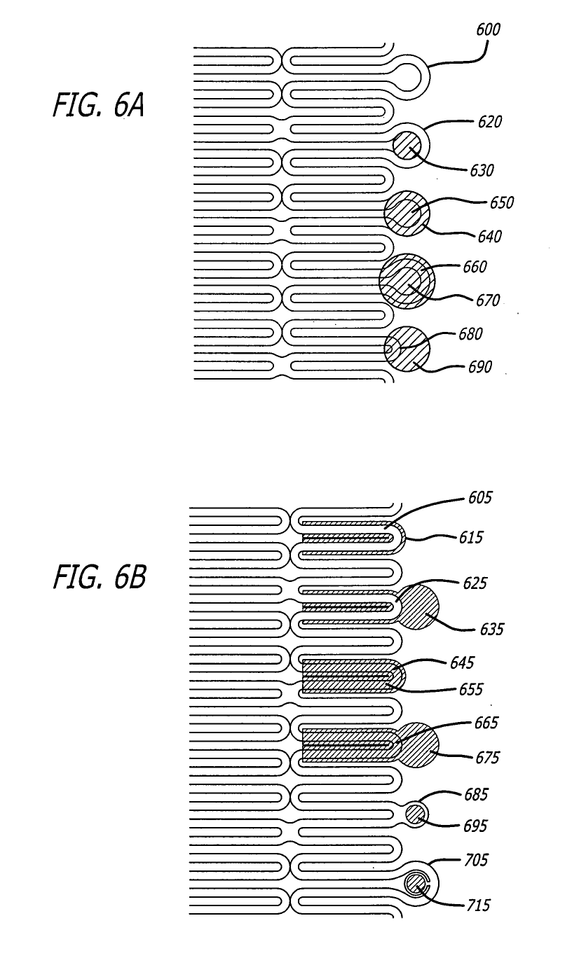

FIGS. 6A and 6B depict end sections of stents with additional degradable material configurations. In FIG. 6A, crowns 600, 620, 640 and 660 comprise bulbed ends. The bulbed end of crown 600 has no degradable feature. The bulbed end of crown 620 includes a degradable material 630 in the form a disc within an end crown 620 formed of a nondegradable material. This depicted embodiment provides another example of an embodiment where a nondegradable framework is filled in with a degradable material. The bulbed end of crown 640 includes a degradable material 650 inside and around the nondegradable end crown 640. The bulbed end of crown 660 includes a degradable material 670 inside, around and extending over the edges of the nondegradable end crown 660. End crown 680 does not include a bulbed end. In this depicted embodiment, degradable material 690 extends more significantly beyond the end of end crown 680.

FIG. 6B depicts additional alternative degradable material configurations around the end crowns of a stent. In this FIG. 6B, end crown 605 includes a degradable material 615 extending over its surface. End crown 625 includes a degradable material 635 over its surface and extending beyond the end of crown 625. End crown 645 is encapsulated within a degradable material 655. End crown 665 is encapsulated within a degradable material 675 that extends beyond the end of crown 665. End crowns 685 and 705 depict two different embodiments of bulbed end crowns with degradable materials 695 and 715 inserted within the crowns as discs. These embodiments provide further examples of nondegradable frameworks filled in with degradable materials.

There are many approaches that can be adopted to create a stent with both degradable and nondegradable portions. For example, a degradable portion of a stent could be formed around a nondegradable portion by insert molding and/or solvent casting. Degradable and nondegradable portions could also be sandwiched together by, without limitation, cladding, press fitting or clamping. FIG. 7 depicts various non-limiting mechanisms that can be used to connect degradable and nondegradable portions of stents according to the present invention. In FIG. 7, section 720 is degradable while section 730 is nondegradable. These two sections of the depicted stent are connected at five points 740, 750, 760, 770 and 780. Connection point 740 depicts attachment by press fit. Connection point 750 depicts attachment by encapsulation of a portion of the nondegradable end crown 755 with degradable material. Connection point 760 depicts attachment using a bonding technique between the degradable and nondegradable portions of the stent. Appropriate bonding techniques include, without limitation, adhesive bonding, cohesive bonding, welding, chemical bonding, solvent bonding, and thermal bonding. Connection point 770 depicts attachment with a mechanical fastener between the degradable and nondegradable portions of the stent. Connection point 780 depicts attachment with an alternative form of a mechanical fastener between the degradable and nondegradable portions of the stent. Non-limiting examples of appropriate mechanical fasteners between degradable and nondegradable portions of the presently disclosed stents include sutures, thread, wire, cable, tape, straps, snaps, barbs, clips, press fit couplings, clamps, screws, bolts, pins and dowels. Additionally, one material can serve as a base material with the second covering it and then being cut away (through, without limitation, laser cutting, stamp cutting or chemical etching) to leave the second material in a different pattern than the first material. Covering of the first material with the second material can be achieved by, without limitation, dipping, casting, molding, wrapping, and spraying.

In addition to providing increased surface area for bioactive material deposition, the degradable tabs of the present invention also provide for temporarily increased mass of the stent. This temporarily increased mass (which is not structurally required) is beneficial for enhanced radiopacity in stent positioning. For example, the degradable tabs or other degradable features placed at different portions along the length of a stent can be used to position the stent appropriately in an area of a vessel bifurcation or other specific treatment sites. Further, the degradable tabs and stent sections of the present invention can lack bioactive materials altogether and only be used for enhanced radiopacity, can include bioactive materials within the degradable material, can have bioactive materials coated onto the surface of the degradable material or can include bioactive materials within and coated onto the surface of the degradable material. Further, nondegradable portions of the stent also can have bioactive materials included on their surface.

As stated earlier, embodiments of the present invention can increase the surface area of a stent at any point along the body or end crowns of the stent. Increasing the surface area of a stent for additional bioactive material deposition and subsequent release at one or more ends of a stent may ease the transition between stented and unstented portions of a vessel by providing for additional bioactive materials that combat restenosis at the transition site.

The tabs of the present invention, whether found within crowns or extending from the ends of crowns can take on a variety of shapes and sizes. Further, the stents of the present invention can be used in any blood vessel, including, for example and without limitation, the coronary vasculature (which includes, without limitation, the right, left common, left anterior descending and circumflex arteries and their branches) and the peripheral vasculature (including without limitation branches of the carotid, aorta, femoral, renal, popliteal, and related arteries). While the stents of the present invention mainly have been described in terms of their use in a blood vessels, they can also be used in other lumens of the body, for example and without limitation, respiratory ducts, gastrointestinal ducts, bile ducts, the urinary system, the digestive tube, and the tubes of the reproductive system in both men and women.

The degradable tabs and sections of the present invention can be formed with a variety of appropriate materials. Some of these materials include, for example and without limitation, degradable polymers such as, without limitation, poly[lactide-co-glycolide], polyanhydrides, and polyorthoesters, whose carboxylic groups are exposed on the external surface as their smooth surface erodes. In addition, polymers containing labile bonds, such as, without limitation, polyanhydrides and polyesters, are well known for their hydrolytic reactivity. Their hydrolytic degradation rates can generally be altered by simple changes in the polymer backbone.

In accordance with the present invention, degradable tabs and sections can be constructed from polymers or monomers using linkages susceptible to biodegradation, such as ester, peptide, anhydride, orthoester, and phosphoester bonds.

Non-limiting examples of degradable components which are hydrolyzable are polymers and oligomers of glycolide, lactide, ε-caprolactone, other a-hydroxy acids, and other biologically degradable polymers that yield materials that are non-toxic or present as normal metabolites in the body. Non-limiting examples of poly(a-hydroxy acid)s include, without limitation, poly(glycolic acid), poly(DL-lactic acid) and poly(L-lactic acid). Other useful materials include, without limitation, poly(amino acids), poly(anhydrides), poly(orthoesters), and poly(phosphoesters). Polylactones such as poly(ε-caprolactone), poly(ε(3 caprolactone), poly(6-valerolactone) and poly(gamma-butyrolactone), for example, also can be used with the present invention.

These described polymers and materials can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

The degradable tabs and sections of the present invention also can include, for example and without limitation, magnesium or magnesium alloys (both hereinafter referred to as magnesium). Magnesium can include bioactive materials, when, for example, it is electroformed with a bioactive material in an electrocodeposition bath. Appropriate procedures for such electroforming are disclosed in, for example, co-pending U.S. patent application Ser. No. 11/220,328 filed on Sep. 6, 2005, which is hereby incorporated by reference for all it contains regarding electrocodeposition and electroforming. Alternatively, magnesium can be bioactive material free and instead have a bioactive material containing polymer applied over it.

Other appropriate materials for use as degradable materials in accordance with the present invention can include hydroxyapatite, aluminum-calcium-phosphorous oxide, bone meal, tricalcium phosphate ceramic implants and glass ceramics with high mechanical strength in the (50−x/2)CaO. SiO(2)-xB(2)O(3) (4.2<or =x <or =17.2) range as described by the School of Materials Science & Engineering, College of Engineering, Seoul National University, Seoul 151-742, Korea, which is incorporated by reference herein for its teachings regarding degradable glass ceramics. These glass ceramics consist of three phases: monoclinic wollastonite, calcium metaborate, and amorphous borosilicate matrix and form an apatite layer on its surface in simulated body fluid and with significant degradation.

Materials incorporated to enhance radiopacity can be, without limitation, mixed, coated, filled, encapsulated, complexed, dyed, and absorbed into a used degradable material. Non-limiting examples of appropriate radiopacity enhancing materials include a cyclic carbonate of ioxilan (IXC) mixed with radiopaque microspheres made from albumin molecules either containing or complexed with a radiopaque substances for clinical use; medical grade calcium sulfate; covalently bound iodine; water soluble contrast agents including, without limitation, metrizamide, iopamidol, iothalamate sodium, iodomide sodium, and meglumine; water insoluble contrast agents including, without limitation, tantalum, tantalum oxide, barium sulfate (PLA 96-BaSO₄), gold, tungsten, and platinum powders; and MRI visible materials including, without limitation, gadolinium oxide.

It is to be understood that the present invention is not limited to the particular embodiments, materials, and examples described herein, as these can vary. Further, the tabs of the present invention can be made of material that is dissimilar to the material or materials that make up the other portions of the stent. It also is to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a stent” or “a tab” is a reference to one or more stents or tabs and includes equivalents thereof known to those skilled in the art and so forth.

Unless defined otherwise, all technical terms used herein have the same meanings as commonly understood by one of ordinary skill in the art to which this invention belongs. Specific methods, devices, and materials are described, although any methods and materials similar or equivalent to those described herein can be used in the practice or testing of the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A stent comprising:
a first end portion, a central portion and a second end portion;
wherein said first end portion, said central portion and said second end portion each comprise one or more sections;
wherein each of said one or more sections comprises a series of struts and crowns; and
wherein at least a part of said one or more sections is degradable.

2. A stent according to claim 1, wherein said degradable part comprises a bioactive material, a radiopacity enhancing material, or both.

3. A stent according to claim 1, wherein said central portion is non-degradable.

4. A stent according to claim 1 wherein the location of said degradable part is selected from the group consisting of within said first end portion; within said central portion; within said second end portion; and combinations thereof.

5. A stent according to claim 1, wherein one or more of said one or more sections in said first end portion are degradable.

6. A stent according to claim 5, wherein one or more sections adjacent to said one or more degradable sections in said first end portion are also degradable.

7. A stent according to claim 5, wherein one or more of said one or more sections of said second end portion are degradable.

8. A stent according to claim 7, wherein one or more sections adjacent to said one or more degradable sections of said second end portion are also degradable.

9. A stent according to claim 5, wherein said one or more degradable sections comprise a bioactive material, a radiopacity enhancing material, or both.

10. A stent according to claim 1, further comprising at least one degradable tab.

11. A stent according to claim 10, wherein said at least one degradable tab comprises a bioactive material, a radiopacity enhancing material, or both.

12. A stent according to claim 10, wherein said first end portion has an end section and said at least one degradable tab extends from an end crown of said end section.

13. A stent according to claim 12, wherein said second end portion has an end section comprising at least one degradable tab extending from an end crown on said end section.

14. A stent according to claim 10 wherein said at least one degradable tab is within a crown.

15. An endoprosthetic device comprising:
a plurality of segments including a proximal end segment, a distal end segment, at least one intermediate segment between said proximal end segment and said distal end segment and a degradable disc.

16. An endoprosthetic device according to claim 15 wherein said degradable disc comprises a bioactive material, a radiopacity enhancing material, or both.

17. An endoprosthetic device according to claim 15 further comprising a plurality of degradable discs each comprising a bioactive material, a radiopacity enhancing material, or both.

18. An endoprosthetic device according to claim 15 wherein:
said plurality of segments are arranged along and define a longitudinal axis; and said proximal end segment has a series of crowns, struts and end crowns and further wherein at least one said end crown has a rim extending longitudinally away from said at least one intermediate segment and wherein said rim is adapted to receive said degradable disc.

19. An endoprosthetic device according to claim 18 further comprising:
a plurality of degradable discs; and
wherein the distal end segment also has a series of crowns, struts and end crowns and further wherein at least one said distal segment end crown has a rim extending longitudinally away from said at least one intermediate segment and wherein said rim is adapted to receive one of said plurality of degradable discs.

20. An endoprosthetic device according to claim 18, wherein said degradable disc comprises a bioactive material, a radiopacity enhancing material, or both.
